(19) United States
(12) Patent Application Publication
(10) Pub. No.: US 2025/0245820 A1
(43) Pub. Date: Jul. 31, 2025

(54) CONTROLLABLE NO-REFERENCE DENOISING OF MEDICAL IMAGES

(71) Applicant: KONINKLIJKE PHILIPS N.V., EINDHOVEN (NL)

(72) Inventors: ELVIRA ZAINULINA, MOSKVA (RU); ALEXEY CHERNYAVSKIY, MOSKVA (RU)

(21) Appl. No.: 18/880,861

(22) PCT Filed: Jul. 4, 2023
(86) PCT No.: PCT/EP2023/068402

§ 371 Date: Jan. 3, 2025

(30) Foreign Application Priority Data
Jul. 6, 2022 (RU) 2022118347

(51) Int. Cl. G06T 7/00; G06T 5/60; G06T 5/70; G16H 30/20

(52) U.S. Cl. CPC G06T 7/0012; G06T 5/60; G06T 5/70; G16H 30/20; G06T 2207/20081; G06T 2207/20084

(57) ABSTRACT

A method for training a machine-learning model for denoising is provided, including retrieving a target image data frame. The target image data frame is one image data frame of a sequence containing imaging data of a subject. The method further includes retrieving at least one prior image data frame and at least one following image data frame of the sequence. Contents of the prior and following image data frames each overlap partially with contents of the target image data frame. The method further includes retrieving acquisition parameters associated with the image data frames of the sequence and generating a prediction for a denoised target image data frame based on the prior and following image data frame. The method trains a machine-learning algorithm based on the prediction and a noise model based on the acquisition parameters. Also provided are a system and denoising method.

### FIELD

The present disclosure generally relates to systems and methods for training and using neural network models for denoising images without the use of reference images during training. In particular, the present disclosure relates to systems and methods for training and using such neural network models in the context of computed tomography (CT) images.

### BACKGROUND

Conventionally, in imaging modalities such as computed tomography, there are effects in the acquisition physics or reconstruction that lead to artifacts, such as noise, in the final image. While low dose computed tomography (LDCT) is widely used in radiology, x-ray dose reduction increases the noise level which affects diagnostic performance.

The best performing techniques for noise mitigation are based on deep learning models trained on pairs of clean-noisy images of the same anatomy. Accordingly, in order to train a denoising algorithm utilizing machine-learning, such as a neural network model, pairs of noisy and noiseless image samples are typically presented to the neural network model, and the network attempts to minimize a cost function by denoising the noisy image to recover a corresponding noiseless ground truth image.

Noiseless images, or clean images, are difficult to obtain, as they typically require a high radiation dose in order to generate images of a high quality. Obtaining such data in a clinical setting would typically take at least two times longer than a regular exam and could increase a patient's exposure to radiation. Further, even if such an approach is taken, paired images may not be ideally aligned because of patient movements which can cause separate artifacts in the denoised images. Accordingly, pairs of images usable for training purposes may be difficult to obtain, particularly in a clinical setting.

Currently, paired datasets are created by adding synthetic noise to real high-quality images. However, such a noise model may fail when underlying mathematical assumptions do not match an acquisition setup. Accordingly, there is a need for denoising techniques that allow for training without clean high dose images being made available.

Further, training of a neural network model for denoising is typically specific to a particular quality of image, where the quality of the image depends on acquisition parameters of the image. Accordingly, an image acquired with half of a typical radiation dose will have a different amount of noise than an image acquired with a quarter of a typical radiation dose.

The neural network model used for denoising is therefore typically specific to a set of acquisition parameter, and a change in parameters used to acquire an image may result in changes in the form, or amount, of artifacts in the corresponding image. As such, a denoising model used to denoise an image obtained using a first set of parameters is less effective when applied to an image acquired using different acquisition parameters, such as a reduced radiation dose in the context of a CT scan. The noise level of the images used during training, therefore, restricts the generalization capabilities of the denoising model trained. For example, if the training set contains images having moderate noise level, the algorithm will not be able to accurately denoise images with high noise levels. Usually, the application of the algorithm will cause over-smoothing or incomplete removal of noise.

In CT imaging, multiple factors, including peak tube voltage, measured in Kilovoltage peak (kVp), tube current, measured in Milliampere-Seconds (mAs), slice thickness, column position, and patient size all may affect the noise level in a reconstructed image. A result is that varying any of these imaging parameters may thereby result in different noise levels, or different artifact profiles, traditionally preventing the generalization of denoising capabilities and requiring a different model to denoise images acquired with such distinct imaging parameters. This limits the applicability of CNN based methods in practical denoising.

Supervised denoising methods rely on the properties of the data used for training. Accordingly, the degree of denoising can be regulated only by choosing appropriate data for training or by other post-processing techniques, such as a weighted combination of an initial noisy image with its noise residual. This technique is known as over-correction.

Current self-supervised denoising methods do not require low-noise reference images, but they possess several weaknesses. They do not use the information contained in sequences of connected images, such as CT projections. Instead, every image is denoised separately, which leads to sub-optimal image quality and longer execution times. Some of the methods require additional data, complicating the performance of the denoising method. Further, like supervised methods, they do not allow to regulate the denoising level.

There is therefore a need for a controllable denoising method that can be trained without clean reference images and that can be used to denoise images acquired with a variety of imaging parameters. There is a further need for a single trained model that can be used to denoise images with different noise levels, including CT images acquired with a lower radiation dose than the images used for training.

### SUMMARY

Systems and methods for denoising medical images are provided in which no reference images to be used as clean images are used for training. The proposed method instead relies on the similarities existing in sequences of connected images and modeling distributions of the clean and noisy data. A special noise module may allow denoising algorithms to better generalize to different noise levels and regulate the degree of denoising by tuning interpretable parameters, manually or automatically. The possibility of adjusting the noise module and training only on noisy data makes the proposed no-reference denoising method rather flexible for application in a clinical setting.

In some embodiments, a method for training a machine-learning model for denoising is provided. The method includes retrieving a target image data frame. The target image data frame is one image data frame of a sequence of image data frames containing imaging data of a subject.

The method further includes retrieving at least one prior image data frame of the sequence of image data frames prior to the target image data frame in the sequence. Contents of the at least one prior image data frame overlap at least partially with the contents of the target image data frame. The method further includes retrieving at least one following image data frame of the sequence of image data frames following the target image data frame in the sequence. Contents of the at least one following image data frame overlap at least partially with contents of the target image data frame.

The method further includes retrieving acquisition parameters associated with the acquisition of the image data frames of the sequence of image data frames and generating a prediction for a denoised target image data frame based on the at least one prior image data frame and the at least one following image data frame.

The method then trains a machine-learning algorithm to denoise the target image data frame based on the prediction for the denoised target image data frame and a noise model based on the acquisition parameters.

In some embodiments, the prediction for the denoised target image data frame is an estimation of the mean and standard deviation of the denoised target image data frame based on a representation of at least one anatomical feature extracted from each of the at least one prior image data frame and the at least one following image data frame. The representations from the at least one prior image data frame and the at least one following image data frame are then fused to form the prediction for the denoised target image data frame.

In some such embodiments, the representations of the at least one anatomical feature are transported between frames using convolutional memory units. Such convolutional memory units may be convolutional long short-term memory units for carrying information between frames of the sequence of image data frames.

In some embodiments, the at least one prior image data frame is a plurality of image data frames in the sequence of image data frames prior to the target image data frame. The at least one following image data frame may similarly be a plurality of image data frames of the sequence of image data frames following the target image data frame.

In some embodiments, the prediction for the denoised target image data frame is output by a trained convolutional neural network provided with the at least one prior image data frame and the at least one following image data frame.

In some embodiments, a loss function for training the machine learning algorithm is based on a distribution of noise expected based on the acquisition parameters. Such a distribution of noise may be based on a Poisson-Gaussian distribution.

In some such embodiments, the training method is repeated for sequences of projection frames obtained using different acquisition parameters, and a tuning variable is extracted or generated for the machine-learning algorithm based on the results associated with different acquisition parameters. In some such embodiments, the tuning variable is trained based on variances in a tube current associated with acquisition of an associated sequence of image data frames.

In some other such embodiments, the tuning variable is a scaling factor that determines how much noise is identified by the machine-learning algorithm to be removed.

In some embodiments, the acquisition parameters are extracted from a DICOM file associated with the sequence of projection frames. In some embodiments, the machine learning algorithm is a convolutional neural network.

In some embodiment, the imaging data is CT imaging data, and each image data frame of the sequence of image data frames is a projection frame. Each projection frame comprises imaging data of the same subject acquired from a different angle.

A denoising method may also be provided in which a machine-learning algorithm is trained in the way discussed above. The method then retrieves a sequence of noisy image data frames including a noisy target image data frame to be denoised. The method then includes selecting a value for the tuning variable based on acquisition parameters of the sequence of noisy image data frames.

The method then includes applying the trained machine-learning algorithm to the sequence of noisy image data frames using the selected value for the tuning variable. The method then generates a first denoised image data frame based on an estimation of a mean and standard deviation based on the sequence of noisy image data frames, a distribution of noise based on the acquisition parameters of the sequence of noisy image data frames, and the noisy target image data frame.

In some such embodiments, the tuning variable is trained to correspond to an acquisition parameter in the training data, and a selected value for the tuning variable is different than an actual value of the corresponding acquisition parameter of the noisy image data frame. The machine-learning algorithm then identifies more noise in the noisy image data frame when using the selected value than when using the actual value.

In some embodiments, the denoising method further includes reconstructing an image based on a plurality of denoised image data frames including the first denoised image data frame, and outputting the reconstructed image to a user.

Also provided is a machine learning training system including a memory that stores a plurality of instructions and processor circuitry that couples to the memory and is configured to execute the instructions to implement the training method discussed above.

### BRIEF DESCRIPTION OF THE DRAWINGS

FIG. 1 is a schematic diagram of a system according to one embodiment of the present disclosure.

FIG. 2 illustrates an exemplary imaging device according to one embodiment of the present disclosure.

FIG. 3 illustrates a pipeline for training a model used for denoising images in accordance with the present disclosure.

FIG. 4 illustrates the use of a model for denoising images in accordance with the present disclosure.

FIG. 5A provides a flowchart illustrating a method for training a model for denoising images in accordance with the present disclosure.

FIG. 5B provides a flowchart illustrating a method for denoising images in accordance with the present disclosure.

FIG. 6 provides a schematic diagram of the use of a tuning variable in a model for denoising images in accordance with the present disclosure.

### DETAILED DESCRIPTION OF THE PREFERRED EMBODIMENTS

The description of illustrative embodiments according to principles of the present disclosure is intended to be read in connection with the accompanying drawings, which are to be considered part of the entire written description. In the description of embodiments of the disclosure disclosed herein, any reference to direction or orientation is merely intended for convenience of description and is not intended in any way to limit the scope of the present disclosure. Relative terms such as “lower,” “upper,” “horizontal,” “vertical,” “above,” “below,” “up,” “down,” “top” and “bottom” as well as derivative thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation unless explicitly indicated as such. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the disclosure are illustrated by reference to the exemplified embodiments. Accordingly, the disclosure expressly should not be limited to such exemplary embodiments illustrating some possible non-limiting combination of features that may exist alone or in other combinations of features: the scope of the disclosure being defined by the claims appended hereto.

This disclosure describes the best mode or modes of practicing the disclosure as presently contemplated. This description is not intended to be understood in a limiting sense, but provides an example of the disclosure presented solely for illustrative purposes by reference to the accompanying drawings to advise one of ordinary skill in the art of the advantages and construction of the disclosure. In the various views of the drawings, like reference characters designate like or similar parts.

It is important to note that the embodiments disclosed are only examples of the many advantageous uses of the innovative teachings herein. In general, statements made in the specification of the present application do not necessarily limit any of the various claimed disclosures. Moreover, some statements may apply to some inventive features but not to others. In general, unless otherwise indicated, singular elements may be in plural and vice versa with no loss of generality.

Generally, images acquired for use in a medical setting require some processing in order to denoise the images. Such denoising is necessary in the medical setting, where images are likely to be used for diagnoses and treatment, as precision and accuracy in such images can improve their usability. Such denoising is typically implemented using machine learning based algorithms, such as convolutional neural networks (CNNs).

CNNs used for denoising require training in order to properly recognize noise in the context of medical imaging. Traditionally, such CNNs would be trained using pairs of images, where each pair comprises a first “noisy” image and a second clean image, where the clean image is used as ground truth. The CNN is then trained to compare the noisy image to the clean image and process the noisy image, so that an output image approximates the clean image. In order to train a CNN in this way, a training set comprising large number of pairs of images are necessary. Further, in order to achieve consistent results, the training set would typically comprise images acquired using a consistent set of acquisition parameters, and a resulting CNN would typically be limited to new images acquired using the same or similar acquisition parameters.

The generation of such a training set is difficult and time consuming, as discussed above. Accordingly, the systems and methods described herein do not require paired images and instead rely on adjacent frames in a sequence of image data frames. Such an approach allows for the recreation of image data in a target frame from image data from adjacent frames in a sequence of image data frames. Such adjacent frames could provide different representations of the contents of the target frame with a different noise distribution. By combining multiple adjacent frames, a prediction may then be generated for the target frame. Such a prediction may then be an estimation of the mean and standard deviation of a denoised version of the target frame. The CNN may then be trained by using the noisy target frame as ground truth data, where a loss function for training is based on the prediction generated based on the multiple adjacent frames.

When training the CNN based on the sequence of images, the noise in CT projections, for example, depends on technical acquisition parameters that can be extracted from an acquisition description coming from, e.g., a DICOM file. A method may then consider a set of acquisition parameters under which the sequence of image data frames was acquired. The acquisition parameters may be used to model an expected noise distribution, which is then used in the generation of the prediction for the target frame. Alternatively, or in combination with such an approach, the model of the expected noise distribution may be used, in combination with the prediction for the target frame to further inform the loss function.

The use of acquisition parameters can make the model more adaptable and, consequently, less dependent on the properties of the dataset used during training. The systems and methods described herein may then be used to create tunable CNNs for denoising image data. In such an embodiment, a denoising model may create an estimation of noise variance based on the acquisition parameters. A noise estimation module may then be trained with the CNN, such that the acquisition parameters are considered in the denoising process. By training the method using sequences of frames acquired using different acquisition parameters, the CNN may then be trained to consider acquisition parameters during the denoising process, thereby allowing the CNN to function across a range of acquisition parameters.

Further, a tunable variable may be extracted from the model by which the noise estimation based on the acquisition parameters can be manipulated. During denoising based on the CNN, the tunable variable may then be used to increase or decrease an amount of noise to be removed from an image.

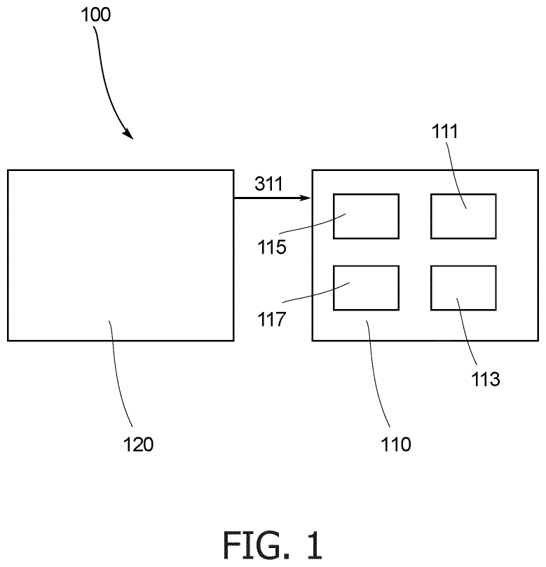

FIG. 1 is a schematic diagram of a system 100 according to one embodiment of the present disclosure. As shown, the system 100 typically includes a processing device 110 and an imaging device 120.

The processing device 110 may apply processing routines to images or measured data, such as projection data, received from the image device 120. The processing device 110 may include a memory 113 and processor circuitry 111. The memory 113 may store a plurality of instructions. The processor circuitry 111 may couple to the memory 113 and may be configured to execute the instructions. The instructions stored in the memory 113 may comprise processing routines, as well as data associated with processing routines, such as machine learning algorithms, and various filters for processing images.

The processing device 110 may further include an input 115 and an output 117. The input 115 may receive information, such as images or measured data, from the imaging device 120. The output 117 may output information, such as filtered images, to a user or a user interface device. The output may include a monitor or display.

In some embodiments, the processing device 110 may relate to the imaging device 120 directly. In alternate embodiments, the processing device 110 may be distinct from the imaging device 120, such that the processing device 110 receives images or measured data for processing by way of a network or other interface at the input 115.

In some embodiments, the imaging device 120 may include an image data processing device, and a spectral or conventional CT scanning unit for generating CT projection data when scanning an object (e.g., a patient).

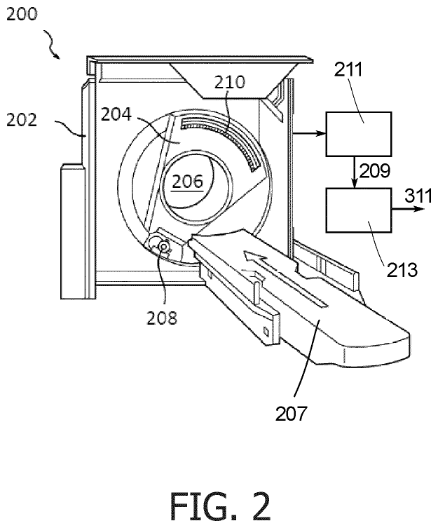

FIG. 2 illustrates an exemplary imaging device 200 according to one embodiment of the present disclosure. It will be understood that while a CT imaging device is shown, and the following discussion is generally in the context of CT images, similar methods may be applied in the context of other imaging devices, and images to which these methods may be applied may be acquired in a wide variety of ways.

In an imaging device in accordance with embodiments of the present disclosure, the CT scanning unit may be adapted for performing multiple axial scans and/or a helical scan of an object in order to generate the CT projection data. Accordingly, the multiple scans may be recorded as a sequence of scans, each containing image data and recorded as an image data frame. In an imaging device in accordance with embodiments of the present disclosure, the CT scanning unit may comprise an energy-resolving photon counting image detector. The CT scanning unit may include a radiation source that emits radiation for traversing the object when acquiring the projection data.

In the example shown in FIG. 2, the CT scanning unit 200, e.g. the Computed Tomography (CT) scanner, may include a stationary gantry 202 and a rotating gantry 204, which may be rotatably supported by the stationary gantry 202. The rotating gantry 204 may rotate about a longitudinal axis around an examination region 206 for the object when acquiring the projection data. The CT scanning unit 200 may include a support 207 to support the patient in the examination region 206 and configured to pass the patient through the examination region during the imaging process.

The CT scanning unit 200 may include a radiation source 208, such as an X-ray tube, which may be supported by and configured to rotate with the rotating gantry 204. The radiation source 208 may include an anode and a cathode. A source voltage applied across the anode and the cathode may accelerate electrons from the cathode to the anode. The electron flow may provide a current flow from the cathode to the anode, such as to produce radiation for traversing the examination region 206.

The CT scanning unit 200 may comprise a detector 210. The detector 210 may subtend an angular arc opposite the examination region 206 relative to the radiation source 208. The detector 210 may include a one- or two-dimensional array of pixels, such as direct conversion detector pixels. The detector 210 may be adapted for detecting radiation traversing the examination region 206 and for generating a signal indicative of an energy thereof.

Generally, the CT scanning unit acquires a sequence of projection frames as the rotating gantry 204 rotates about the patient. Accordingly, depending on the amount of gantry movement between frame, each acquired frame of projection data overlaps to some extent with adjacent frames, and consists of imaging data of the same subject, i.e., the patient, acquired at a different angle. In the context of this application, the frames may comprise different types of data, such as raw signal data, tomographic projection data, or image data at various stages of processing. All of these types of data comprise imaging data, and each frame of the sequence of frames thereby contains some type of imaging data.

Accordingly, in some embodiments, the imaging data in the frames may be processed to different degrees prior to transmitting the data to the processing device 110. The CT scanning unit 200 may therefore include generators 211 and 213. The generator 211 may generate tomographic projection data 209 based on the signal from the detector 210. The generator 213 may receive the tomographic projection data 209 and, in some embodiments, generate a sequence of raw image data frames 311 of the object based on the tomographic projection data 209. In some embodiments, the tomographic projection data 209 may be provided to the input 115 of the processing device 110, while in other embodiments the sequence of raw image data frames 311 is provided to the input of the processing device.

As discussed below in more detail, in addition to the sequence of image data frames provided to the input 115 of the processing device 110, the image device 120 may also provide data defining acquisition parameters associated with the sequence of image frames. Such acquisition parameters may include incident photon flux distribution over detector column positions and tube currents and/or voltages used. Such acquisition parameters may be provided in the context of the Digital Imaging and Communications in Medicine (DICOM) standard, and may be transmitted with the underlying sequence of image data frames, for example. As such, the acquisition parameters may be extracted from a DICOM file.

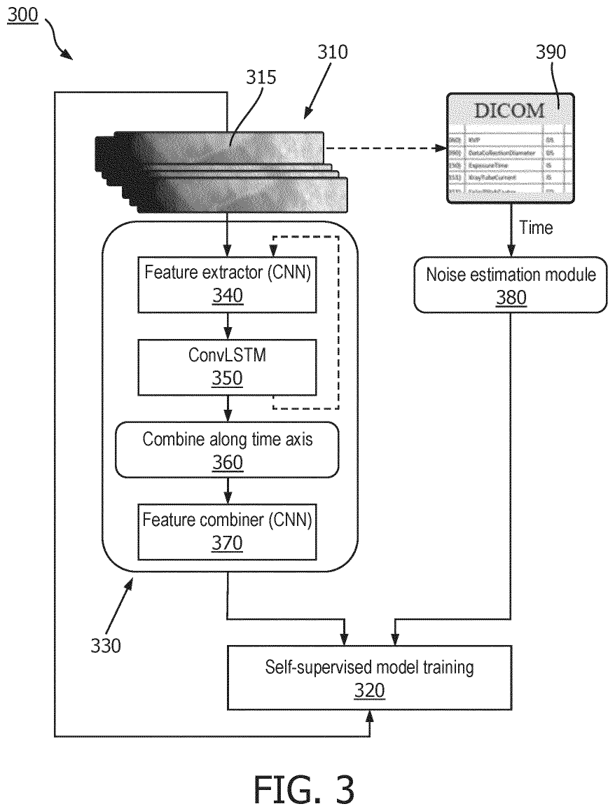

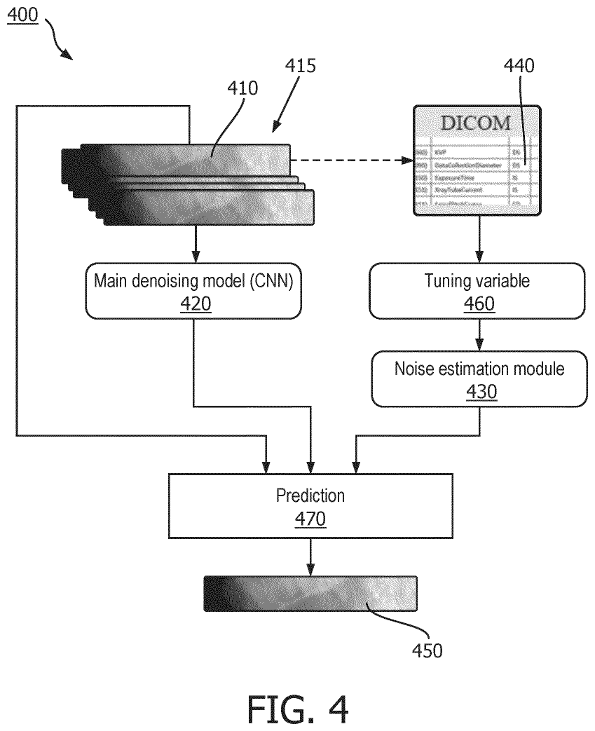

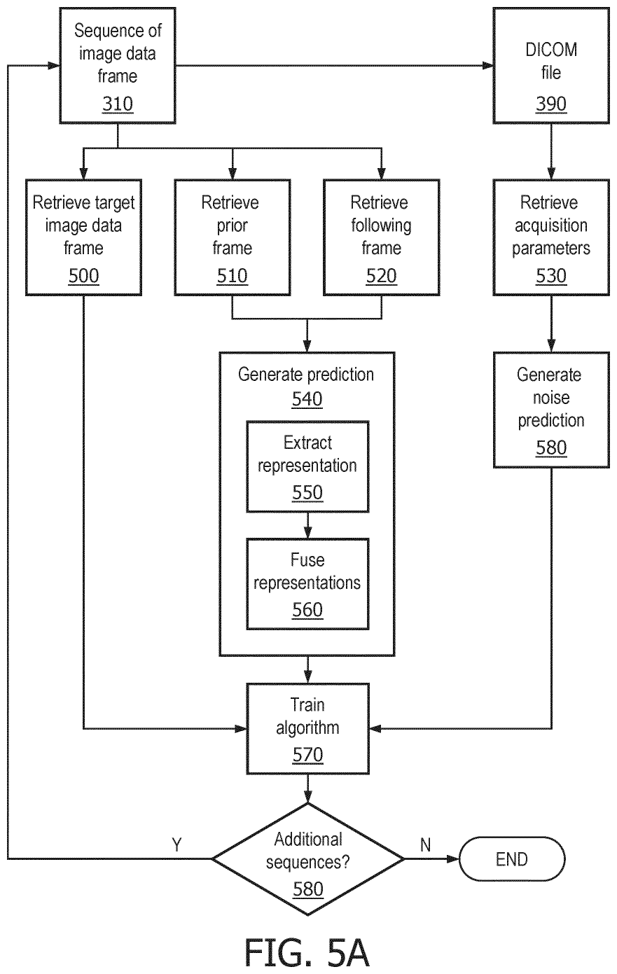

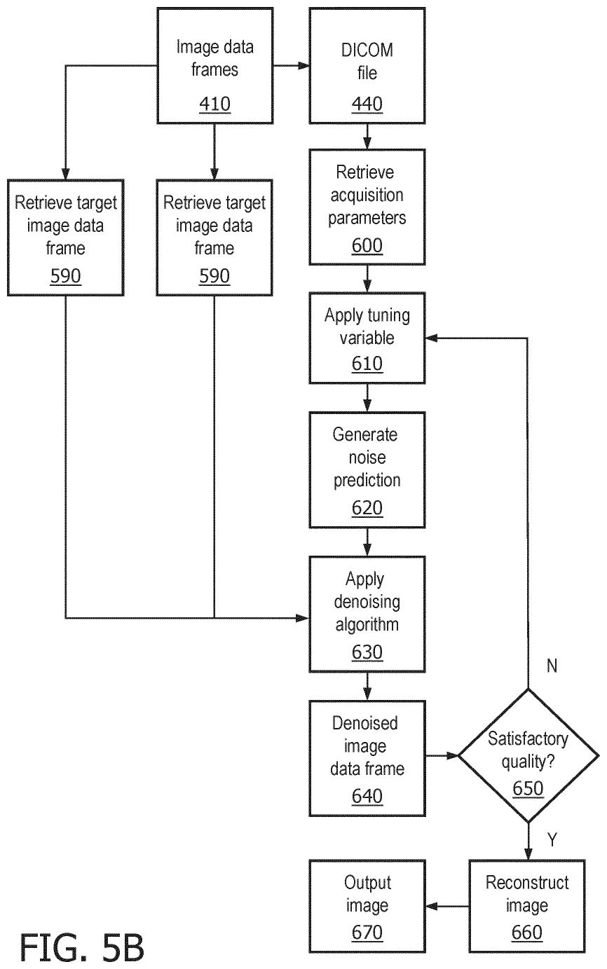

FIG. 3 illustrates a pipeline 300 for training a model used for denoising images in accordance with the present disclosure. FIG. 4 illustrates the use 400 of a model for denoising images in accordance with the present disclosure. As shown, and as discussed in more detail below with respect to FIG. 5, a method for training the model will first retrieve or be provided with a sequence of image data frames 310 which may then be used for denoising any one of those image data frames. A target image data frame 315 is then provided directly to a model training module 320, while a separate prediction module 330 predicts the contents of the target image data frame.

The prediction module 330 receives image data from at least one image data frame prior to and at least one image data frame following a target image data frame 315 and generates, from those frames, a prediction of the contents of the target image data frame. In doing so, the prediction module 330 extracts contents of the image data frames, such as anatomical features of the subject of the image data, from the image data frames provided to it by a feature extractor module 340. This feature extractor module 340 may be a discrete and separately trained learning algorithm, such as a CNN.

Contents extracted from the image data frames are then considered across multiple frames using, for example, convolutional memory units, such as convolutional long short-term memory units (ConvLSTM) 350. The features extracted are then combined along the time axis (at 360) and fused by a feature combiner module 370.

The fused features may be anatomical features of a subject of the image data and may be used to predict the contents of the target image data frame, already provided to the model training module 320. The output of the prediction module 330 is then a prediction for the target image data frame. For example, the prediction may be an estimation of the mean and standard deviation of the denoised target frame. The model training module 320 then trains a machine-learning algorithm, such as a CNN, using the noisy target image data frame 315 as ground truth. In the embodiment shown, the CNN trained by the model training module 320 is the denoising module 330.

In some embodiments, such as that shown, a noise estimation module 380 may be provided to predict a noise distribution in the prediction. The noise estimation module 380, discussed in more detail below, may then provide a prediction in the form of a noise model based on acquisition parameters 390 associated with the sequence of image data frames 310, which may take the form of a DICOM file or metadata associated with the underlying image data. As discussed in more detail below, the noise estimation module 380 may itself be a machine learning algorithm, such as a CNN, and the model training module 320 may also train the noise estimation module 380.

In such embodiments, the prediction provided by the noise estimation module 380 is provided to the model training module 320 along with the prediction for the target image data frame for better defining a loss function for use with the target image data frame 315 as ground truth data.

Once trained, the model 400 may be deployed as shown in FIG. 4. Accordingly, optionally, a target image data frame 410 and at least one prior image data frame and one following image data frame extracted from a sequence 415 of image data frames may be provided to a denoising model 420, and a noise estimate may be generated by a noise estimation module 430 based on the acquisition parameters 440, and may then be used by a prediction engine 470 to output a denoised image 450 derived from the target image data frame 410.

As discussed in detail below, the noise estimation module 430 may be provided with a tuning variable 460 derived during training 300. Such a tuning variable may be analogous to a physical characteristic of image acquisition, as recorded in the acquisition parameters 440 and may be user selectable. As such, a user may select a value for the tuning variable different than the actual characteristic stored in the corresponding acquisition parameters 440 in order to scale the output of the noise estimation module. Alternatively, the tuning variable 460 may be a general value associated with noise level in the model and may be scaled without regard for a corresponding physical characteristic.

FIG. 5A provides a flowchart illustrating a method for training a model for denoising images in accordance with the present disclosure.

As shown, and as discussed in the context of FIG. 3, the method retrieves a target image data frame (500) to be considered. The target image data frame is one image data frame of a sequence of image data frames 310 containing imaging data of a subject. The imaging data may be extracted from a CT scanning system, such as that discussed above with respect to FIG. 2. In such an embodiment, the image data frame is a projection frame and is part of a sequence of CT projection frames. Accordingly, each projection frame typically comprises imaging data of the same subject acquired from a different angle.

The method then proceeds to retrieve at least one prior image data frame (510) of the sequence of image data frames prior to the target image data frame (retrieved at 500) in the sequence. The contents of the at least one prior image data frame overlap, at least partially, with the contents of the target image data frame. The method further retrieves at least one following image data frame (520) of the sequence of image data frames following the target image data frame in the sequence. The contents of the at least one following image data frame overlap at least partially with the contents of the target image data frame.

In this way, the method acquires image data frames prior to and following the target frame in sequence. The prior and following frames are typically adjacent frames, and in addition to overlapping at least partially with the target frame, they overlap at least partially with each other. In this way, the prior and following frames carry at least some of the same content. When frames are described as overlapping, it is understood that the overlap may be in terms of an image associated with or generated from the image data frame or the actual subject of the image data frame. For example, where images are acquired in a linear process, images associated with the image data frames may comprise adjacent images having some overlapping and, therefore, identical image content. However, in cases where images are acquired axially or helically, as in the case of CT projection frames, the image content would not overlap but the subject of the images would overlap, as the images would typically comprise the same subject, acquired from a different angle. Where an angle between the projections acquired is small, the contents of the image data frames are said to overlap. For example, in some embodiments, the image data frames may be said to overlap so long as the same feature side is visible, and in such embodiments, image data frames may overlap so long as the angle between the projections is less than 90 degrees. In other embodiments, a degree of overlap can be measured by evaluating differences between images. This may be by way of a structural similarity index measure (SSIM) or mean squared error (MSE), for example.

In some embodiments, the method retrieves a plurality of prior image data frames and retrieves a plurality of image data frames in the sequence of image data frames prior to the target image data frame. Similarly, the method may retrieve a plurality of following image data frames, thereby retrieving a plurality of image data frames in the sequence of image data frames following the target image data frame. Each of the frames retrieved in this manner would have at least some overlapping content, and the number of adjacent frames retrieved may depend on how much the content of the frames overlap, as well as the computational and memory capacity of the device implementing the method. Such values may be regulated by a user as well.

While the flowchart illustrates the separate retrieval of each of the frames identified, it is understood that the method may instead retrieve a sequence of image data frames 310 from an imaging system as a file or may have access to a database containing such a sequence, and the method would then identify a target image data frame 315 in that data along with the prior and following image data frames.

The method then retrieves acquisition parameters (530) associated with the acquisition of the image data frames of the sequence of image data frames 310. As discussed above, such acquisition parameters may be provided as a DICOM file 390 associated with or drawn from the sequence of image data frames 310 or may be included as metadata in the image data frames themselves.

The method then proceeds to generate (540) a prediction for a denoised target image data frame based on the at least one prior image data frame (retrieved at 510) and the at least one following image data frame (retrieved at 520). Such a prediction may take the form of an estimation of the mean and standard deviation of a clean version of the target data frame 315.

In some embodiments, the method generates the prediction (at 540) for the denoised target image data frame based on a representation of at least one anatomical feature extracted from each of the at least one prior image data frame and the at least one following image data frame. The representations may be extracted (550) from the prior and following image data frames by a feature extractor module 340 and the representation extracted from each image data frame may be fused (560) by a feature combiner module 370 to form the prediction for the denoised target image data frame.

In some such embodiments, the representations of the at least one anatomical feature are transported between frames using convolutional memory units, which may be convolutional long short-term memory units 350 for carrying information between frames of the sequence of image data frames.

In some embodiments, multiple machine-learning algorithms, such as CNNs, may be implemented at different steps of the method. For example, the feature extractor module 340 may implement a CNN to extract features (at 550), such as anatomical features from various image data frames. Similarly, the feature combiner module 340 may implement a CNN to fuse feature representations together (at 560) to form the prediction. Similarly, the prediction for the denoised target image data frame may be generated and output by a single trained convolutional neural network provided with the at least one prior image data frame and the at least one following image data frame. The prediction for the denoised target image data frame may take the form of the mean and standard deviation of a denoised version of the target frame

A self-supervised model training module 320 is then provided with the prediction for the denoised target image data frame, the target image data frame, and a noise model based on the acquisition parameters (retrieved at 530) and trains the prediction module 330 (570) to denoise the target image data frame 315 based on the provided data. The prediction module 330 may be a CNN. The training process may utilize the noisy target data frame 315 as ground truth and may use the prediction (generated at 540) as a basis for a loss function.

In some embodiments, a loss function used to train the machine learning algorithm is based on a distribution of noise expected based on the acquisition parameters. In such embodiments, after the acquisition parameters are retrieved (at 530), a noise model comprising a prediction for a noise distribution is generated (580) at a noise estimation module 380 based on those parameters. Such a prediction for a noise distribution may be utilized in creating the loss function used for training (at 570) and may be simultaneously trained or fine tuned by the training process.

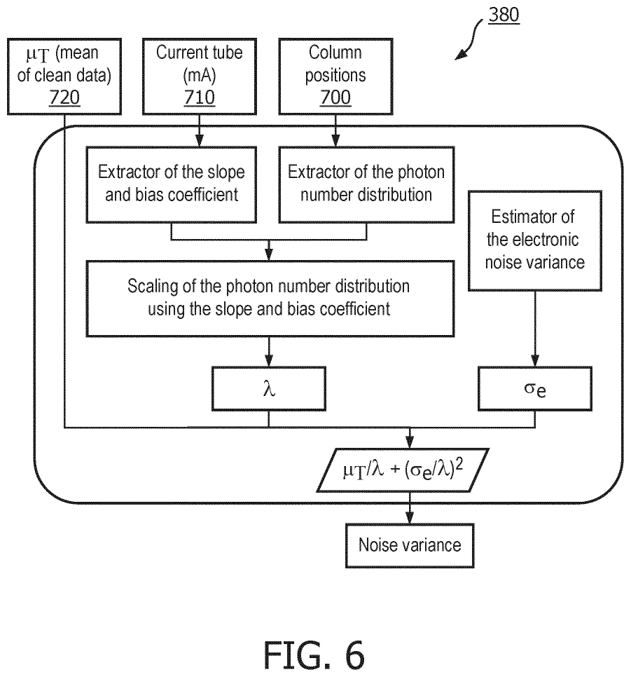

Additional details related to the generation of the noise model by the noise estimation module are discussed in more detail below with respect to FIG. 6.

The training method described herein is typically repeated for a large number of sequences of image data frames. Such repeated sequences may include sequences obtained using different acquisition parameters. Accordingly, after training the algorithm using a sequence of image data frames 310, the method determines if additional sequences are available for training (580). If so, the method proceeds to retrieve target image data frames (at 500), retrieve prior frames (at 510) and retrieve following frames (at 520) from additional sequences of image data frames 310.

Each sequence of image data frames 310 is typically provided with distinct DICOM files 390 defining the associated acquisition parameters. Accordingly, the method may, for each iteration, retrieve such acquisition parameters (at 530). In this way, the training of the algorithm (at 580) may take into account the acquisition parameters detailed in the DICOM file 390.

As part of the generation of the noise prediction (at 580), the generated model is typically based on details drawn from the DICOM file, as discussed below in reference to FIG. 6. As such, each of the details drawn from the DICOM file effectively tunes the model output as a noise prediction (at 580). During the training of the algorithm, the method may thereby associate at least one of the details drawn from the DICOM file with a designated tuning or scaling variable which may be tuned manually during model inference once the model is trained.

Accordingly, a tuning variable may then be extracted or generated for the machine-learning algorithm based on the results associated with different acquisition parameters. As noted above, the tuning variable may be trained based on and thereby associated with specific variables identified in the DICOM file. For example, the tuning variable may be based on variances in a tube current associated with the acquisition of an image or associated sequence of image data frames.

The tuning variable may be a scaling factor that determines how much noise identified by the machine-learning algorithm is to be removed. Accordingly, the model output as a noise prediction (580) may generate a prediction of the form that noise may take, and the tuning variable may then determine how much noise taking that form is to be removed from a target image data frame.

Such tuning and the expected form of noise predicted by the noise estimation module 380 is discussed in more detail below with respect to FIG. 6.

FIG. 5B provides a flowchart illustrating a method for denoising images in accordance with the present disclosure.

Once the machine-learning algorithm, or CNN, is trained, and no more additional sequences are provided for training (at 580), the machine-learning algorithm may be used for denoising images.

Accordingly, the method may retrieve a target noisy image data frame 410 to be denoised (590) along with at least one prior image data frame and at least one following image data frame (595). The method separately retrieves acquisition parameters associated with the noisy image data frame (600). Such acquisition parameters may be drawn from an associated DICOM file 440 associated with or drawn from the image data frame 410.

As discussed above, a tuning variable may be extracted or generated during the training process described with respect to FIG. 5A. As such, a tuning variable may be applied to or associated with the acquisition parameters (610) during model inference. A value for the tuning variable may be selected and applied (at 610) based on the acquisition parameters of the noisy image data frame. For example, the tuning variable may be associated with a detail drawn from the DICOM file and may be set accordingly (at 610).

Once the tuning variable is set (at 610), the method may proceed to generate a noise prediction on that basis (620) and apply the trained machine-learning denoising algorithm (630) to the sequence of noisy image data frames, including, optionally, the target noisy image data frame 410 and the at least one prior image data frame and at least one following image data frame. The method then generates an estimation of the mean and standard deviation of a denoised target image data frame and operates on that estimation combined with the noise prediction and the target image data frame using the selected value for the tuning variable.

The method may then predicts a clean image data frame for the target image data frame to generate a first denoised image data frame (640).

In some embodiments, the first denoised image data frame (generated at 640) may be initially reviewed prior to presenting to a user to determine if quality of the denoising method is satisfactory (650). If so, the first denoised image data frame (generated at 640) may be output to a user. If not, a reviewer may modify the tuning variable (at 610) in order to improve the quality of the first denoised image data frame. In some embodiments, the user himself reviews the first denoised image data frame and determines whether to modify the applied tuning variable (at 610) and again process the image data frame.

As discussed above, the tuning variable may be trained to correspond to an acquisition parameter in the training data. As such, after modification (at 610), the selected value for the tuning variable may be different than the actual value of the corresponding acquisition parameter associated with the noisy image data frame. Where the tuning variable functions to apply a scaling factor, the selected variable may lead the machine-learning algorithm to identify and remove more noise in the noisy image data frame when using the selected value than when using the actual value for the corresponding acquisition parameter.

For example, where the tuning variable is a proxy for an acquisition tube current, a lower tube current typically corresponds to higher noise levels in an image. Accordingly, if a higher value is used for the tuning variable (at 610), the less noise will be removed from the image data frame and the resulting data will be more accurate. However, if a lower value is used for the tuning variable, denoising will be more severe and the resulting data will be cleaner, but at the expense of some anatomical details that might be smoothed out. Accordingly, a user, such as a clinician, may determine whether cleaner or more accurate output is preferable depending on the particular diagnostic task at hand.

In some embodiments, a user may initially apply a selected tuning variable (at 610) to the method without first reviewing output. This may then remove more noise from the noisy image data frame being processed than default values would.

Once quality of the denoising method is determined to be satisfactory (at 650), the method may proceed to reconstruct an image from the image data file (660). Such reconstruction may be necessary where the image data frames are, for example, projection frames from a CT scan that are processed prior to reconstruction. In some embodiments, reconstruction of the image may be based on a plurality of denoised image data frames including the first denoised image data frame. Further, the reconstruction may be a volume or other composition of images rather than a single image. The method may then conclude by outputting the reconstructed image or volume to the user (670).

FIG. 6 illustrates the estimation of noise in an image in the noise estimation module 380 of FIG. 3.

The discussion that follows relates to noise in CT projections, but similar noise estimation methods may be implemented in other imaging modalities. Noise in CT projections can be approximated by a mixed Poisson-Gaussian distribution with parameters that can be obtained from the description of the acquisition process. Such a distribution takes the form:

$$\tilde{T} \sim Normal ( {μ}_{T} , {σ}_{T}^{2} + \frac{{μ}_{T}}{λ} + \frac{{σ}_{e}^{2}}{{λ}^{2}} ) , T = exp ( - p ) ,$$

where p is normalized projection data without noise, and T is transmission data without noise. T can be approximated by the Gaussian distribution Normal (μ_{T}, σ_{T}²) and noise can be modeled by the Normal distribution

$$Normal ( 0 , \frac{{μ}_{T}}{λ} + \frac{{σ}_{e}^{2}}{{λ}^{2}} ) ,$$

\tilde{T} is the noisy transmission data,
λ is the incident number of photons,
σₑ² is electronic noise variance.

The electronic noise variance can be obtained from the measurements of the dark current, while the incident number of photons can be estimated from air scans. However, such values are typically not readily available. Accordingly, in the method described herein, the provided noise estimation module 380 removes the need to know these parameters exactly. Instead, they can be estimated by an optimization algorithm while training the main denoising model. Nevertheless, the theory helps to make pre-estimations. If CT scanners use bowtie filtering and automatic exposure control, the noise parameter λ corresponding to the incident flux of photons should depend on the detector column position 700 and the tube current 710. The trainable noise estimation module 380 then takes CT acquisition properties, such as from the DICOM file 390 discussed above, as input and produces an estimate of the noise variance

$${σ}_{n}^{2} = \frac{{μ}_{T}}{λ} + \frac{{σ}_{e}^{2}}{{λ}^{2}} .$$

In our main embodiment, the noise estimation module is implemented as a small neural network that predicts the noise variance from the mean of the clean transmission data μ_{T} 720, detector column positions 700 and tube currents (mAs) 710 used.

The tube current 710 may then be used to extract the slope and bias coefficient and the column positions 700 may be used to extract a prediction of the photon number distribution. The extracted slope and bias coefficient may then be used to scale the expected photon number distribution to derive a prediction for the incident number of photons λ. The method may separately estimate electronic noise variance to derive a prediction for σₑ². Which may then be used to estimate the noise variance σₑ².

The main denoising model 320 and the noise estimation module 380 can be trained together, or the parameters of the noise estimation module can be pre-computed. The training is performed using the available noisy CT projections 310 and their acquisition parameters. If the test data have other noise properties (e.g., bowtie filtering is not used), the noise estimation module 380 can be tuned in accordance with such properties, while the main denoising model 320 can remain unchanged. In such a scenario, where acquisition parameters are changed, either both the denoising model 320 and the noise estimation module 380, or the noise estimation module alone 380, can be re-trained directly on the new data.

It will be understood that although the methods described herein are described in the context of CT scan images, various imaging technology, including various medical imaging technologies are contemplated, and images generated using a wide variety of imaging technologies can be effectively denoised using the methods described herein. Because the method described includes a discrete noise model, modifications of the noise model allows the method to be used for processing other medical image sequences. Accordingly, relevant modalities can include, for example, dynamic positron emission tomography (PET), dynamic magnetic resonance (MR), microscopic fluorescence images, ultrasonograms, and others.

The methods according to the present disclosure may be implemented on a computer as a computer implemented method, or in dedicated hardware, or in a combination of both. Executable code for a method according to the present disclosure may be stored on a computer program product. Examples of computer program products include memory devices, optical storage devices, integrated circuits, servers, online software, etc. Preferably, the computer program product may include non-transitory program code stored on a computer readable medium for performing a method according to the present disclosure when said program product is executed on a computer. In an embodiment, the computer program may include computer program code adapted to perform all the steps of a method according to the present disclosure when the computer program is run on a computer. The computer program may be embodied on a computer readable medium.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

All examples and conditional language recited herein are intended for pedagogical purposes to aid the reader in understanding the principles of the disclosure and the concepts contributed by the inventor to furthering the art, and are to be construed as being without limitation to such specifically recited examples and conditions. Moreover, all statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

What is claimed is:

1. A method for training a machine-learning model for denoising, comprising:
retrieving a target image data frame, the target image data frame being one image data frame of a sequence of image data frames containing imaging data of a subject;
retrieving at least one prior image data frame of the sequence of image data frames prior to the target image data frame in the sequence, wherein contents of the at least one prior image data frame overlap at least partially with the contents of target image data frame;
retrieving at least one following image data frame of the sequence of image data frames following the target image data frame in the sequence, wherein contents of the at least one following image data frame overlap at least partially with contents of the target image data frame;
retrieving acquisition parameters associated with the acquisition of the image data frames of the sequence of image data frames;
generating a prediction for a denoised target image data frame based on the at least one prior image data frame and the at least one following image data frame;
training a machine-learning algorithm to denoise the target image data frame based on the prediction for the denoised target image data frame and a noise model based on the acquisition parameters.

2. The method of claim 1, wherein the prediction for the denoised target image data frame is an estimation of the mean and standard deviation of the denoised target image data frame based on a representation of at least one anatomical feature extracted from each of the at least one prior image data frame and the at least one following image data frame and wherein the representations from the at least one prior image data frame and the at least one following image data frame are fused to form the prediction for the denoised target image data frame.

3. The method of claim 2, wherein the representations of the at least one anatomical feature are transported between frames using convolutional memory units.

4. The method of claim 3, wherein the convolutional memory units are convolutional long short-term memory units for carrying information between frames of the sequence of image data frames.

5. The method of claim 2, wherein the at least one prior image data frame is a plurality of image data frames in the sequence of image data frames prior to the target image data frame and wherein the at least one following image data frame is a plurality of image data frames of the sequence of image data frames following the target image data frame.

6. The method of claim 2, wherein the prediction for the denoised target image data frame is output by a trained convolutional neural network provided with the at least one prior image data frame and the at least one following image data frame.

7. The method of claim 1, wherein a loss function for training the machine learning algorithm is based on a distribution of noise expected based on the acquisition parameters.

8. The method of claim 7, wherein the training method is repeated for sequences of projection frames obtained using different acquisition parameters, and wherein a tuning variable is extracted or generated for the machine-learning algorithm based on the results associated with different acquisition parameters.

9. The method of claim 8, wherein the tuning variable is trained based on variances in a tube current associated with acquisition of an associated sequence of image data frames.

10. The method of claim 8, wherein the tuning variable is a scaling factor that determines how much noise identified by the machine-learning algorithm is to be removed.

11. The method of claim 7, wherein the expected distribution of noise is based on a Poisson-Gaussian distribution.

12. The method of claim 1 wherein the acquisition parameters are extracted from a DICOM file associated with the sequence of projection frames.

13. The method of claim 1, wherein the machine learning algorithm is a convolutional neural network.

14. The method of claim 1, wherein the imaging data is CT imaging data, and wherein each image data frame of the sequence of image data frames is a projection frame, and wherein each projection frame comprises imaging data of the same subject acquired from a different angle.

15. A denoising method comprising:
performing the method of claim 8;
retrieving a sequence of noisy image data frames including a noisy target image data frame to be denoised;
selecting a value for the tuning variable based on acquisition parameters of the sequence of noisy image data frames;
applying the trained machine-learning algorithm to the sequence of noisy image data frames using the selected value for the tuning variable;
generating a first denoised image data frame based on an estimation of a mean and standard deviation based on the sequence of noisy image data frames, a distribution of noise based on the acquisition parameters of the sequence of noisy image data frames, and the noisy target image data frame.

16. The denoising method of claim 15, wherein the tuning variable is trained to correspond to an acquisition parameter in the training data, and wherein a selected value for the tuning variable is different than an actual value of the corresponding acquisition parameter of the noisy image data frame, and wherein the machine-learning algorithm identifies more noise in the noisy image data frame when using the selected value than when using the actual value.

17. A machine learning training system comprising:
a memory that stores a plurality of instructions; and
processor circuitry that couples to the memory and is configured to execute the instructions to:
retrieve a plurality of image data frames comprising a sequence of image data frames containing imaging data of a subject;
identify a target image data frame of the sequence of image data frames;
generate a prediction for a denoised target image data frame based on at least one prior image data frame of the sequence of image data frames prior to the target image data frame in the sequence and at least one following image data frame following the target image data frame in the sequence, wherein each of the at least one prior image data frame and the at least one following image data frame overlap at least partially with the target image data frame;
retrieve acquisition parameters associated with the acquisition of the image data frames of the sequence of image data frames;
train a machine-learning algorithm to denoise the target image data frame based on the prediction for the denoised target image data frame and a noise model based on the acquisition parameters.

18. The system of claim 17, wherein a loss function for training the machine learning algorithm is based on a distribution of noise expected based on the acquisition parameters.

19. The system of claim 18, wherein the training method is repeated for sequences of image data frames obtained using different acquisition parameters, and wherein a tuning variable is extracted or generated for the machine learning algorithm based on the results associated with different acquisition parameters.

20. The system of claim 18, wherein the imaging data is CT imaging data, and wherein each image data frame of the sequence of image data frames is a projection frame, and wherein each projection frame comprises imaging data of the same subject acquired from a different angle.
